SEQUENCE LISTING (6 sequences)

SEQ ID NO: 1 - DNA, 1758 bp, Yeast
atgggcttgttaacgaaagtagccacgtcgagacaattctctacaacgagatccgttgccaagaaactaaacaagtattcctacatcatcactgaaccaaagggccaaggtgcctcccaggccatgctttacgctaccgggttcaagaaggatgacttccaaaagcctcaggttggtgtcggttcatgctggtggtctggtaacccatgcaacatgcatctgttggacttgaacaacagatgttcccaatccatcgaaaaggctggcctaaaggccatgcagttcaacaccatcggtgtttctgacggtatctccatgggtaccaagggtatgagatactctttgcaaagtagagagatcattgcggactcgtttgaaaccatcatgatggctcagcattacgatgccaacatcgccatcccatcttgtgacaagaacatgcccggtgtcatgatggccatgggtagacacaatagaccctccatcatggtctacggtggtaccatcttgcccggtcacccaacctgcgggtcctccaagatctccaaaaacatcgacattgtctcagctttccaatcttacggtgagtatatctccaagcagttcactgaagaagagagagaagatgtcgtggaacacgcctgccctggtcctggttcttgtggtggtatgtacaccgccaacacaatggcctctgccgcagaagtcctaggtttaaccattccaaactcctcttccttccccgccgtgtccaagcagaagttggccgagtgtgacaatattggtgaatacatcaagacaacaatggaattgggcattttgccccgtgacatcctcaccaaggaggcctttgaaaatgccattacttacgtcgttgccacaggtgggtccaccaatgctgttttgcatctggttgccgttgctcattccgcgggtgttaagttgtctccagatgatttccaaagaatcagtaacaccacaccattgatcggtgacttcaagccttccggtaaatacgtcatggctgacttgatcaacgtcggtggtacacaatccgtgattaagtacctatacgaaaacaacatgttacatggtaacaccatgactgtcaccggtgacactttggcagaacgtgccaagaaggcaccaagcttgcctgaaggccaagagatcatcaaacctctttcccatccaatcaagaccagcggtcacttacaaatcttatacggttcattggcgccaggtggtgccgtgggtaaaattaccggtaaggaaggtacttacttcaaaggtaaggcccgtgtctacgaagaagaaggtgcctttattgaagctttggaaaagggcgaaatcaagaagggtgaaaaaactgttgttattatcagatacgaaggtccaagaggtgctccaggtatgcctgaaatgctaaagccttcttctgctctaatggggtacggtttaggtaaagatgtcgcattgttgactgatggtagattctccggtggttctcatgggttcttgatcggtcacattgtcccagaagccgctgaaggtggacctattggtcttgtgaaagacggtgacgaaatcattatcgatgctgacaacaacaagattgatttgttgatctccgaacaggaaatgtctcaacgtaagcaaaactgggtcgctcctccacctcgttacacaagaggtactttatccaaatatgctaagttggtttccaacgcttccagcggttgtgtcctagatgcttga

SEQ ID NO: 2 - protein, 585 aa, Yeast
MGLLTKVATSRQFSTTRSVAKKLNKYSYIITEPKGQGASQAMLYATGFKKDDFQKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKQKLAECDNIGEYIKTTMELGILPRDILTKEAFENAITYVVATGGSTNAVLHLVAVAHSAGVKLSPDDFQRISNTTPLIGDFKPSGKYVMADLINVGGTQSVIKYLYENNMLHGNTMTVTGDTLAERAKKAPSLPEGQEIIKPLSHPIKTSGHLQILYGSLAPGGAVGKITGKEGTYFKGKARVYEEEGAFIEALEKGEIKKGEKTVVIIRYEGPRGAPGMPEMLKPSSALMGYGLGKDVALLTDGRFSGGSHGFLIGHIVPEAAEGGPIGLVKDGDEIIIDADNNKIDLLISEQEMSQRKQNWVAPPPRYTRGTLSKYAKLVSNASSGCVLDA

SEQ ID NO: 3 - DNA, 40 bp, Artificial
  Primer
gagctcatagcggccatgggcttgttaacgaaagtagcca

SEQ ID NO: 4 - DNA, 42 bp, Artificial
  Primer
ggatcctatgcggccgcataataaatatttatatatataaag

SEQ ID NO: 5 - DNA, 80 bp, Artificial
  Primer
atgggcttgttaacgaaagttgctacatctagacaattctctacaacgagatgcgttgcaccttgacagtcttgacgtgc

SEQ ID NO: 6 - DNA, 80 bp, Artificial
  Primer
tcaagcatctaaaacacaaccgttggaagcgttggaaaccaacttagcatacttggatagcgcacttaacttcgcatctg

(19) United States
(12) Patent Application Publication
(10) Pub. No.: US 2009/0148555 A1
(43) Pub. Date: Jun. 11, 2009

(54) Dihydroxy-Acid Dehydratase Gene and Use Thereof

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, Osaka, Osaka-shi (JP)

(21) Appl. No.: 11/988,453

(22) PCT Filed: Aug. 11, 2006
(86) PCT No.: PCT/JP2006/316206

§ 371 Date: Jan. 8, 2008

(30) Foreign Application Priority Data
Aug. 12, 2005 (JP) 2005-235013

(51) Int. Cl. C12G 1/00; C12N 15/31; C07K 14/39; C12N 15/81; C12Q 1/68; C12N 1/19; C12C 11/00

(52) U.S. Cl. 426/15; 536/23.74; 530/350; 435/320.1; 435/254.2; 426/11; 426/16; 426/592; 435/6

(57) ABSTRACT

The present invention relates to an dihydroxy-acid dehydratase gene and use thereof, in particular, a brewery yeast for producing alcoholic beverages having superior flavor, alcoholic beverages produced with said yeast, and a method for producing said beverages. More particularly, the present invention relates to a yeast whose level of producing total vicinal diketones, especially level of producing diacetyl, that are responsible off-flavor of the product, is reduced by amplifying the expression level of ILV3 gene encoding brewery yeast dihydroxy-acid dehydratase ILV3p, particularly non-ScILV3 gene specific to lager brewing yeast, and to a method for producing alcoholic beverages with said yeast.

### TECHNICAL FIELD

The present invention relates to an dihydroxy-acid dehydratase gene and use thereof, in particular, a brewery yeast for producing alcoholic beverages with superior flavor, alcoholic beverages produced with said yeast, and a method for producing said beverages. More particularly, the present invention relates to a yeast, whose amount of production of diketones, especially diacetyl, that are responsible for off-flavors in products, is reduced by amplifying expression level of ILV3 gene encoding yeast dihydroxy-acid dehydratase ILV3p, especially the non-ScILV3 gene specific to a lager brewing yeast, and to a method for producing alcoholic beverages with said yeast.

### BACKGROUND ART

Flavor of Diacetyl, hereinafter also referred to as “DA”, is a representative off-flavor in brewed alcoholic beverages such as beer, sake and wine and so on among flavoring substances of alcoholic beverages. DA flavor, which is also referred to as “butter flavor” or “sweaty flavor” in beer, “tsuwari-ka”, which means a nauseating flavor, in sake, occurs when vicinal diketones, hereinafter also referred to as “VDK”, mainly DA, are present above certain threshold levels in products. The threshold level is said to be 0.1 ppm (parts per million) in beer (Journal of the Institute of Brewing, 76, 486 (1979)).

VDK in alcoholic beverages can be broadly divided into DA and 2,3-pentanedione, herein after referred to as “PD”. DA and PD are formed by non-enzymatic reactions, which yeasts are not involved in, of α-acetolactic-acid and α-acetohydroxybutyric-acid as precursors which are intermediate in biosynthesis of valine and isoleucine, respectively.

According to these information, VDKs (i.e., DA and PD) and their precursors α-acetohydroxy-acids (i.e., α-acetolactic-acid and α-acetohydroxybutyric-acid) are thought to be the compounds which can impart DA flavors to products. Accordingly, breeding of yeasts which steadily reduces these compounds makes manufacturing control of alcoholic beverages easy as well as expands capability of developing new products.

A method for suppressing production of DA by using rice-malt-yeast culture containing low level of pyruvic acid which is a precursor of acetohydroxy-acids in production of sake in, for example, Japanese Patent Application Laid-Open No. 2001-204457. It is also reported that production of VDKs are reduced in valine, leucine and isoleucine auxotrophic yeast in beer production. However, the yeast has not come into practical use since auxotrophic strains tend to show retarded growth/fermentation. Japanese Patent Application Laid-Open NO. 2002-291465 discloses a method obtaining variant strains sensitive to analogues of the branched amino acids described above, and selecting DA low accumulating strains from the variant strains. Genetically engineered yeasts derived from laboratory-designed yeasts whose amount of expression of ILV5 gene is regulated is reported in Journal of American Society of Brewing Chemists, Proceeding, 81-84 (1987), and also genetically engineered yeast whose amount of expression of ILV3 gene is regulated is reported in European Brewery Convention, Proceedings, of the 21st EBC congress, Madrid, 553-560 (1987). The enzymatic activity of the acetohydroxy-acid reductoisomerase encoded by ILV5 gene is increased 5 to 7-fold, and the amount of production of VDKs are reduced to about 40% in the case.

Besides, the enzymatic activity of the dihydroxy-acid dehydratase encoded by ILV3 is increased 5 to 6-fold. On the contrary, no significant reduction of the amount of production of VDKs was observed. However, any influence on practical beer brewing is analyzed in the two reports described above where synthetic media are used. On the other hand, Villa et al. reported in Journal of American Society of Brewing Chemists, 53; 49-53 (1995), that plasmid amplification of the gene products of ILV5, ILV3 or tandem ILV5+ILV3 in brewer's yeast resulted in VDK decreases of 70, 40 and 60% respectively, when compared to that of normal brewer's yeast on practical beer brewing.

Also, Dulieu et al. proposed a method converting α-acetolactic-acid, which served as a precursor of DA, rapidly to acetoin using α-acetolactate decarboxylase in European Brewery Convention, Proceedings of the 26th EBC congress, Maastricht, 455-460 (1997). However, addition of enzymes to fermented broth is prohibited for tax reasons in Japan. Genetically engineered yeasts using DNA strands encoding α-acetolactate decarboxylase are reported in both Japanese Patent Application Laid-Open Nos. H2-265488 and H07-171.

### DISCLOSURE OF INVENTION

Under circumstances described above, there were demands for developing a method for producing alcoholic beverages capable of reducing the production of VDKs (vicinal diketones), especially DA (diacetyl).

To solve the problems described above, the present inventors made exhaustive studies, and as a result succeeded in identifying and isolating a gene encoding dihydroxy-acid dehydratase from lager brewing yeast.

Thus, the present invention relates to a novel dihydroxy-acid dehydratase gene existing specifically in a lager brewing yeast, to a protein encoded by said gene, to a transformed yeast in which the expression of said gene is controlled, to a method for controlling the level of VDKs, especially the level of DA, in a product by using a yeast in which the expression of said gene is controlled. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO:1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 with one or more amino acids thereof being deleted, substituted, inserted and/or added, and having an dihydroxy-acid dehydratase activity;

(d) a polynucleotide comprising a polynucleotide encoding a protein having an amino acid sequence having 60% or higher identity with the amino acid sequence of SEQ ID NO:2, and having an dihydroxy-acid dehydratase activity;

(e) a polynucleotide comprising a polynucleotide which hybridizes to a polynucleotide consisting of a nucleotide sequence complementary to the nucleotide sequence of SEQ ID NO:1 under stringent conditions, and which encodes a protein having an dihydroxy-acid dehydratase activity; and

(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having an dihydroxy-acid dehydratase activity.

(2) The polynucleotide of (1) above selected from the group consisting of:

(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has an dihydroxy-acid dehydratase activity;

(h) a polynucleotide encoding a protein having 90% or higher identity with the amino acid sequence of SEQ ID NO: 2, and having an dihydroxy-acid dehydratase activity; and

(i) a polynucleotide which hybridizes to SEQ ID NO: 1 or which hybridizes to a nucleotide sequence complementary to the nucleotide sequence of SEQ ID NO: 1 wider stringent conditions, and which encodes a protein having an dihydroxy-acid dehydratase activity.

(3) The polynucleotide of (1) above comprising a polynucleotide consisting of SEQ ID NO: 1.

(4) The polynucleotide of (1) above comprising a polynucleotide encoding a protein consisting of SEQ ID NO: 2.

(5) The polynucleotide of any one of (1) to (4) above, wherein the polynucleotide is DNA.

(6) A protein encoded by the polynucleotide of any one of (1) to (5) above.

(7) A vector comprising the polynucleotide of any one of (1) to (5) above.

(7a) The vector of (7) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(8) A yeast, wherein the vector of (78) above is introduced.

(9) The yeast of (8) above, wherein a capability of producing total vicinal diketones or a capability of producing total diacetyl is reduced by introducing the vector of (7) above.

(10) The yeast of (8) above, wherein a capability of producing total vicinal diketones or a capability of producing total diacetyl is reduced by increasing an expression level of the protein of (6) above.

(11) A method for producing an alcoholic beverage by using the yeast of any one of (8) through (10) above.

(12) The method for producing an alcoholic beverage of (11) above, wherein the brewed alcoholic beverage is a malt beverage.

(13) The method for producing an alcoholic beverage of (11) above, wherein the brewed alcoholic beverage is wine.

(14) An alcoholic beverage produced by the method of any one of (11) through (13) above.

(15) A method for assessing a test yeast for its capability of producing total vicinal diketones or capability of producing total diacetyl, comprising using a primer or a probe designed based on a nucleotide sequence of a dihydroxy-acid dehydratase gene having the nucleotide sequence of SEQ ID NO: 1.

(15a) A method for selecting a yeast having a reduced capability of producing total vicinal diketones or capability of producing total diacetyl by using the method described in (15) above.

(15b) A method for producing an alcoholic beverage (for example, beer) by using the yeast selected with the method described in (15a) above.

(16) A method for assessing a test yeast for its capability of producing total vicinal diketones or capability of producing total diacetyl, comprising: culturing a test yeast; and measuring an expression level of a dihydroxy-acid dehydratase gene having the nucleotide sequence of SEQ ID NO: 1.

(16a) A method for selecting a yeast having a reduced capability of producing total vicinal diketones or capability of producing total diacetyl, which comprises assessing a test yeast by the method described in (16) above and selecting a yeast having a high expression level of dihydroxy-acid dehydratase gene.

(16b) A method for producing an alcoholic beverage (for example, beer) by using the yeast selected with the method in (16a) above.

(17) A method for selecting a yeast, comprising: culturing test yeasts; quantifying the protein of (6) above or measuring an expression level of a dihydroxy-acid dehydratase gene having the nucleotide sequence of SEQ ID NO: 1; and selecting a test yeast having said protein amount or said gene expression level according to a target capability of producing total vicinal diketones or capability of producing total diacetyl.

(17a) A method for selecting a yeast, comprising: culturing test yeasts; quantifying capability of producing total vicinal diketones or capability of producing total diacetyl or activity of an dihydroxy-acid dehydratase; and selecting a test yeast having a target capability of producing total vicinal diketones or capability of producing total diacetyl or activity of dihydroxy-acid dehydratase.

(18) The method for selecting a yeast of (17) above, comprising: culturing a reference yeast and test yeasts; measuring an expression level of a dihydroxy-acid dehydratase gene having the nucleotide sequence of SEQ ID NO: 1 in each yeast; and selecting a test yeast having the gene expressed higher than that in the reference yeast.

(19) The method for selecting a yeast of (17) above comprising: culturing a reference yeast and test yeasts; quantifying the protein of (6) above in each yeast; and selecting a test yeast having said protein for a larger amount than that in the reference yeast.

(20) A method for producing an alcoholic beverage comprising: conducting fermentation for producing an alcoholic beverage using the yeast according to any one of (8) to (10) or a yeast selected by the method according to any one of (17) to (19); and reducing the production amount of total vicinal diketones or the production amount of total diacetyl.

### BRIEF DESCRIPTION OF DRAWINGS

FIG. 1 shows the cell growth with time upon test brew of beer. The horizontal axis represents fermentation time while the vertical axis represents optical density at 660 nm (OD660).

FIG. 2 shows the extract consumption with time upon beer brewing testing. The horizontal axis represents fermentation time while the vertical axis represents apparent extract concentration (w/w %).

FIG. 3 shows the expression behavior of non-ScILV3 gene in yeasts upon test brew of beer. The horizontal axis represents fermentation time while the vertical axis represents the brightness of detected signal.

FIG. 4 shows the results of complementation test of nonScILV3 using ILV3 gene-disrupted strain.

a) This figure shows that ILV3 gene disruption causes auxotrophy for valine, leucine and isoleucine. The parent strain and ILV3 gene-disrupted strain were cultured for 3 days at 30° C. on (-Leu, Ile, Val) plate medium.

b) This figure shows that introduction of nonScILV3 into ILV3 gene-disrupted strain makes the strain non-auxotrophic. The parent strain and ILV3 gene-disrupted strain X2180-1A(ilv3::nat1) were cultured for 3 days at 30° C. on SC (-Leu, Ile, Val) plate medium containing 300 mg/L geneticine and 50 mg/L nourseothricin.

### BEST MODES FOR CARRYING OUT THE INVENTION

The present inventors isolated and identified non-ScILV3 gene encoding an dihydroxy-acid dehydratase unique to lager brewing yeast based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2.

### 1. Polynucleotide of the Invention

First of all, the present invention provides (a) a polynucleotide comprising a polynucleotide of the nucleotide sequence of SEQ ID NO:1; and (b) a polynucleotide comprising a polynucleotide encoding a protein of the amino acid sequence of SEQ ID NO:2. The polynucleotide can be DNA or RNA.

The target polynucleotide of the present invention is not limited to the polynucleotide encoding an dihydroxy-acid dehydratase derived from lager brewing yeast and may include other polynucleotides encoding proteins having equivalent functions to said protein. Proteins with equivalent functions include, for example, (c) a protein of an amino acid sequence of SEQ ID NO: 2 with one or more amino acids thereof being deleted, substituted, inserted and/or added and having an dihydroxy-acid dehydratase activity.

Such proteins include a protein consisting of an amino acid sequence of SEQ ID NO: with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having an dihydroxy-acid dehydratase activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO: 2, and having an dihydroxy-acid dehydratase activity. In general, the percentage identity is preferably higher.

Dihydroxy-acid dehydratase activity may be measured, for example, by a method of Kiritani et al. as described in Methods Enzymol., 17: 755-764 (1970).

Furthermore, the present invention also contemplates (6) a polynucleotide comprising a polynucleotide which hybridizes to a polynucleotide consisting of a nucleotide sequence complementary to the nucleotide sequence of SEQ ID NO: 1 under stringent conditions and which encodes a protein having an dihydroxy-acid dehydratase activity; and (D) a polynucleotide comprising a polynucleotide which hybridizes to a polynucleotide complementary to a nucleotide sequence of encoding a protein of SEQ ID NO: 2 under stringent conditions, and which encodes a protein having an dihydroxy-acid dehydratase activity.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or polynucleotide encoding the amino acid sequence of SEQ ID NO: 2 as a probe. The hybridization method may be a method described, for example, in Molecular Cloning 3rd Ed., Current Protocols in Molecular Biology, John Wiley & Sons 1987-1997.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 50% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 950% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO: 2 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (i) above. A preferred protein of the present invention comprises an amino acid sequence of SEQ ID NO:2 with one or several amino acids thereof being deleted, substituted, inserted and/or added, and has an dihydroxy-acid dehydratase activity.

Such protein includes those having an amino acid sequence of SEQ ID NO: 2 with amino acid residues thereof of the number mentioned above being deleted, substituted, inserted and/or added and having an dihydroxy-acid dehydratase activity. In addition, such protein includes those having homology of about 60% or more, preferably about 70% or more, more preferably about 80% or more, further more preferably about 90% or more, or the most preferably about 95% or more as described above with the amino acid sequence of SEQ ID NO: 2 and having an dihydroxy-acid dehydratase activity.

Such proteins may be obtained by employing site-directed mutation described, for example, in Molecular Cloning 3rd Ed., Current Protocols in Molecular Biology, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides (such as DNA) described in (a) to (i) above. Generally, the vector of the present invention comprises an expression cassette including as components (x) a promoter that can transcribe in a yeast cell; (y) a polynucleotide (such as DNA) described in any of (a) to (i) above that is linked to the promoter in sense or antisense direction; and (z) a signal that functions in the yeast with respect to transcription termination and polyadenylation of RNA molecule.

A vector introduced in the yeast may be any of a multicopy type (YEp type), a single copy type (YCp type), or a chromosome integration type (YIp type). For example, YEp24 (J. R. Broach et al., Experimental Manipulation of Gene Expression, Academic Press, New York, 83, 1983) is known as a YEp type vector, YCp50 (M. D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the brewery yeast and they have no influence on the concentration of constituents such as amino acid and extract in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake. Specifically, yeasts such as genus Saccharomyces may be used. According to the present invention, a lager brewing yeast, for example, Saccharomyces pastorianus W34/70, Saccharomyces carlsbergensis NCYC453 or NCYC456, or Saccharomyces cerevisiae NBRC1951, NBRC1952, NBRC1953 or NBRC1954 may be used. In addition, wine yeasts such as wine yeasts #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth Enzym., 194: 182 (1990)), a spheroplast method (Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), Methods in Yeast Genetics, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York, 117 (1979)), etc.) such that OD600 nm will be 1 to 6. This culture yeast is collected by centrifugation, washed and pre-treated with alkali ion metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in Molecular Cloning 3rd Ed., and Methods in Yeast Genetics, A Laboratory Manual (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to reduce the level of VDKs, especially DA, of desired alcoholic beverages, and produce alcoholic beverages having enhanced flavor. In addition, yeasts to be selected by the yeast assessment method of the present invention can also be used. The target alcoholic beverages include, for example, but not limited to beer, sparkling liquor (happoushu) such as a beer-taste beverage, wine, whisky, sake and the like.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages with an decreased level of VDKs, especially DA. Thus, according to the present invention, alcoholic beverages with enhanced flavor can be produced using the existing facility without increasing the cost.

### 5. Yeast Assessment Method of the Invention

The present invention relates to a method for assessing a test yeast for its capability of producing total vicinal diketones or capability of producing total diacetyl by using a primer or a probe designed based on a nucleotide sequence of a dihydroxy-acid dehydratase gene having the nucleotide sequence of SEQ ID NO:1. General techniques for such assessment method is known and is described in, for example, WO01/040514, Japanese Laid-Open Patent Application No. 8-205900 or the like. This assessment method is described in below.

First, genome of a test yeast is prepared. For this preparation, any known method such as Hereford method or potassium acetate method may be used (e.g., Methods in Yeast Genetics, Cold Spring Harbor Laboratory Press, 130 (1990)). Using a primer or a probe designed based on a nucleotide sequence (preferably, ORF sequence) of the dihydroxy-acid dehydratase gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the capability of producing total vicinal diketones or capability of producing total diacetyl of the yeast as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the dihydroxy-acid dehydratase gene having the nucleotide sequence of SEQ ID NO: 1 to assess the test yeast for its capability of producing total vicinal diketones or capability of producing total diacetyl. In this case, the test yeast is cultured and then mRNA or a protein resulting from the dihydroxy-acid dehydratase gene is quantified. The quantification of mRNA or protein may be carried out by employing a known technique. For example, mRNA may be quantified, by Northern hybridization or quantitative RT-PCR, while protein may be quantified, for example, by Western blotting (Current Protocols in Molecular Biology, John Wiley & Soils 1994-2003).

Furthermore, test yeasts are cultured and expression levels of the gene of the present invention having the nucleotide sequence of SEQ ID NO: 1 are measured to select a test yeast with the gene expression level according to the target capability of producing total vicinal diketones or capability of producing total diacetyl, thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the dihydroxy-acid dehydratase gene having the nucleotide sequence of SEQ ID NO: 1 is measured in each yeast. By selecting a test yeast with the gene expressed higher than that in the reference yeast, a yeast suitable for brewing alcoholic beverages can be selected.

Alternatively, test yeasts are cultured and a yeast with a lower capability of producing total vicinal diketones or capability of producing total diacetyl, or a higher activity of dihydroxy-acid dehydratase is selected, thereby selecting a yeast suitable for brewing desired alcoholic beverages.

In these cases, the test yeasts or the reference yeast may be, for example, a yeast introduced with the vector of the invention, a yeast with amplified expression of the gene of the present invention described above, a yeast with amplified expression of the protein of the present invention described above, an artificially mutated yeast or a naturally mutated yeast. Total amount of vicinal diketones may be quantified by a method described in Drews et al., Mon. fur Brau., 34, 1966. Total amount of diacetyl may be quantified by a method, for example, described in J. Agric. Food Chem. 50(13):3647-53, 2002. Dihydroxy-acid dehydratase activity may be measured, for example, by a method of Kiritani et al. as described in Methods Enzymol., 17: 755-764 (1970). The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g., Yasuji Oshima Ed., Biochemistry Experiments vol. 39, Yeast Molecular Genetic Experiments, pp. 67-75, JSSP).

In addition, examples of yeasts used as the reference yeast or the test yeasts include any yeasts that can be used for brewing, for example, brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954 may be used. Further, whisky yeasts such as Saccharomyces cerevisiae NCYC90; wine yeasts such as wine yeasts #1, 3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Dihydroxy-Acid Dehydratase Gene (non-ScILV3)

A specific novel dihydroxy-acid dehydratase gene (non-ScILV3) gene (SEQ ID NO: 1) from a lager brewing yeast were found, as a result of a search utilizing the comparison database described in Japanese Patent Application Laid-Open No. 2004-283169. Based on the acquired nucleotide sequence information, primers non-ScILV3_F (SEQ ID NO: 3) and non-ScILV3_R (SEQ ID NO: 4) were designed to amplify the full-length genes, respectively. PCR was carried out using chromosomal DNA of a genome sequencing strain, Saccharomyces pastorianus Weihenstephan 34/70 strain, as a template to obtain DNA fragments including the full-length gene of non-ScILV3.

The thus-obtained non-ScILV3 gene fragment was inserted into pCR2.1-TOPO vector (manufactured by Invitrogen Corporation) by TA cloning. The nucleotide sequences of non-ScILV3 gene were analyzed according to Sanger's method (F. Sanger, Science, 214: 1215, 1981) to confirm the nucleotide sequence.

### Example 2

### Analysis of Expression of Non-ScILV3 Gene During Test Brew of Beer

A test brew of beer was conducted using a lager brewing yeast, Saccharomyces pastorianus Weiherstephan 34/70 strain and then mRNA extracted from a beer yeast fungal body during fermentation was detected by a yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast input | 12.8 × 10⁶ cells/mL |

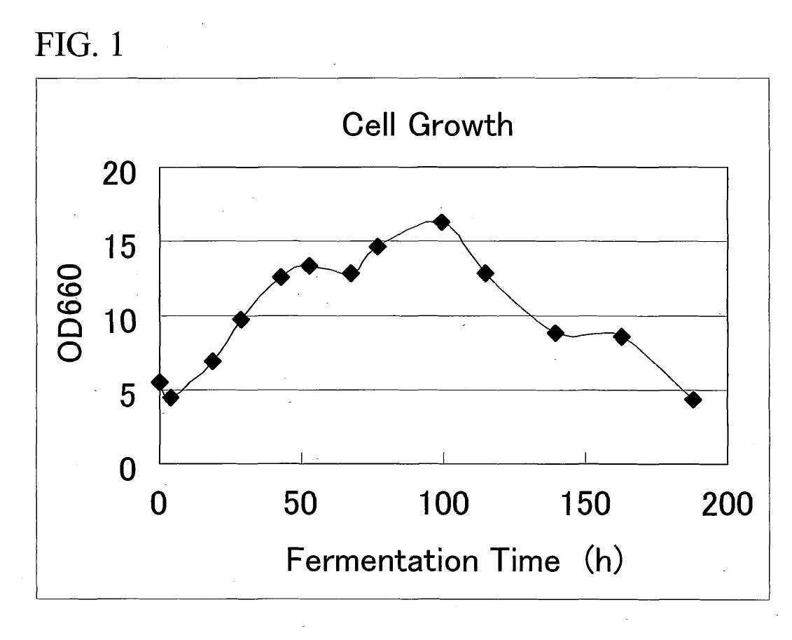

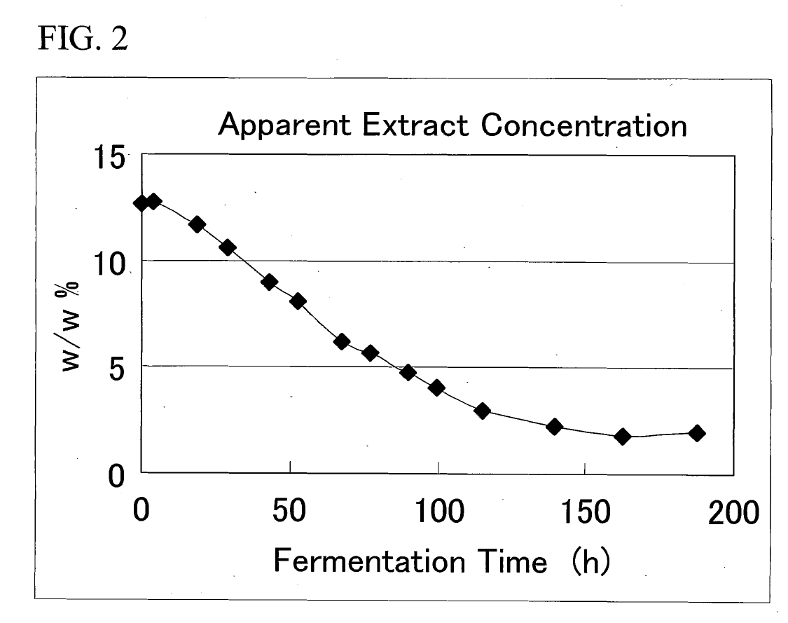

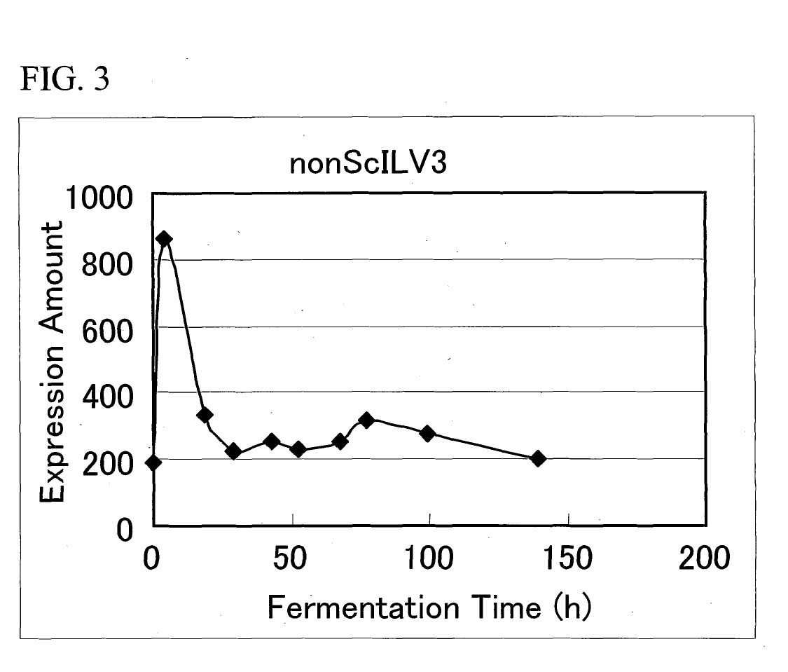

Sampling of fermentation liquid was performed with time, and variation with time of yeast growth amount (FIG. 1) and apparent extract concentration (FIG. 2) was observed. Simultaneously, yeast fungal bodies were sampled to prepare mRNA, and the prepared mRNA was labeled with biotin and was hybridized to a beer yeast DNA microarray described in Japanese Patent Application Laid-Open No. 2004-283169. The signal was detected using GCOS; GeneChip Operating Software 1.0 (manufactured by Affymetrix Co.). Expression pattern of non-ScILV3 gene is shown in FIG. 3. As a result, it was confirmed that non-ScILV3 gene was expressed in the general beer fermentation.

### Example 3

### Complementation Test of Non-ScILV3 Gene Using Laboratory-Designed Yeast Strain

The function of the product of nonScILV3 gene as a dihydroxy-acid dehydratase was confirmed using a laboratory-designed yeast strain whose endogenous ILV3 gene had been disrupted.

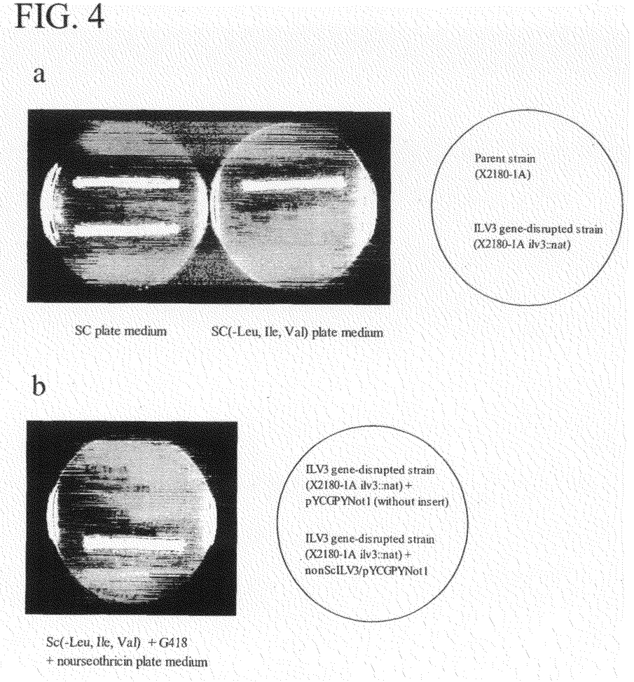

A fragment for disrupting ILV3 gene was prepared by PCR using a plasmid (pAG25 (nat1)) containing a drug resistant marker as a template according to a method described in Coldstein 15, et al., Yeast. 15, 1541 (1999). The sequence of primer utilized are represented by SEQ ID Nos: 5 and 6. S cerevisiae X2180-1A strain was transformed using the fragment by a method described in Japanese Patent Application Laid-Open No. 07-303475, and selected with YPD plate medium (1% yeast extract, 2% polypeptone, 2% glucose, 2% agar) containing 50 mg/L nourseothricin. Resultant ILV3 gene-disrupted strain was inoculated on SC plate medium (0.67% yeast nitrogen base without amino acids, 0.2% amino acid mixture, 2% glucose, 2% agar) and SC plate medium without valine, leucine and isoleucine (0.67% yeast nitrogen base without amino acids, 0.2% amino acid mixture (excepting valine, leucine and isoleucine), 2% glucose, 2% agar). Then the strains were cultured for 3 days at 30° C. to investigate their growth. The ILV3 gene-disrupted strain was not grew on the SC plate medium without valine, leucine and isoleucine as shown in FIG. 4 and Table 1. It was confirmed that the strain was branched amino acid auxotrophy.

Then, a DNA fragment containing whole coding region of the protein was prepared by digesting the nonScILV3/pCR2.1-TOPO described in Example 1 using restriction enzymes SacI and NotI. This fragment was linked to pYCGPYNot treated with restriction enzymes SacI and NotIA, thereby a nonScILV3 constitutive expression vector nonScILV3/pYCGPYNot was constructed. The pYCGPYNot is a Ycp type yeast expression vector. The introduced gene was constitutively expressed by the promoter of a pyruvate kinase gene PYK1. A geneticine-resistant gene G418^{r} was included as a selective marker for yeast. Ampicillin-resistant gene Amp^{r} was also included as a selective marker for E. coli.

The resultant expression vector was transformed to ILV3 gene-disrupted strain (X21180-1A ilv3::nat1). The high expression of nonScILV3 in the transformant was confirmed by RT-PCR. A pYCGPYNot introduced strain without insert was prepared as a control. These strains were evaluated in the same way using SC plate medium without valine, leucine and isoleucine. As a result, growth of the nonScILV3 introduced strain was observed (FIG. 4, Table 1). It was proved that the introduction of nonScILV3 makes the strain non-auxotrophic for branched amino acids. That is to say, the products of the nonScILV3 gene was proved to act as dihydroxy-acid dehydratase.

TABLE 1

| | Growth on SC |
|---|---|
| | (-Leu, Ile, Val) |
| Strain | plate medium |
| Parent strain (X2180-1A) | Grown |
| ILV3 gene-disrupted strain (X2180-1A ilv3::nat) | Not grown |
| ILV3 gene-disrupted strain (X2180-1A ilv3::nat) + | Not grown |
| pYCGPYNot1 (without insert) | |
| ILV3 gene-disrupted strain (X2180-1A ilv3::nat) + | Grown |
| nonScILV3/pYCGPYNot1 | |

### Example 4

### Constitutive Expression of Non-ScILV3 Gene

The constitutive expression vector prepared in Example 3 is used to transform Saccharomyces pastorianus Weihenstephan 34/70 strain according to the method described in Japanese Patent Application Laid-Open No. 07-303475. The transformant is selected with YPD plate medium (1% yeast extract, 2% polypeptone, 2%, glucose, 2% agar) containing 300 mg/L geneticine.

### Example 5

### Analysis of Amount of VDKs Produced During Test Brew of Beer

The parent strain and non-ScILV3 high expression strain obtained in Example 4, are used to carry out fermentation test under the following conditions.

| | |
|---|---|
| Wort extract concentration | 12% |
| Wort content | 1 L |
| Wort dissolved oxygen concentration | 8 ppm |
| Fermentation temperature | 15° C., constant |
| Yeast input | 5 g wet yeast fungal body/L Wort |

The fermentation broth is sampled with time to observe the cell growth (OD660) and extract consumption with time. Quantification of the total VDKs in the fermentation broth is carried out by reacting VDKs (DA and PD) with hydroxylamine to produce glyoxime derivatives, then measuring absorbance of complexes formed from the reaction of resultant glyoxime derivatives and divalent ferric ions (Drews et al., Mon. fur Brau., 34, 1966). The precursors Q-acetolactic-acid and α-acetohydroxybutyric-acid are previously converted to DA and PD, respectively, to quantify the total VDKs including them.

### INDUSTRIAL APPLICABILITY

According to the method for producing alcoholic beverages of the present invention, because of reduction of the production amount of VDKs, especially DA, which are responsible for off-flavors in products, alcoholic beverages with superior flavor can be produced.

1. A polynucleotide selected from the group consisting of:
(a) a polynucleotide comprising a polynucleotide consisting of the nucleotide sequence of SEQ ID NO:1;
(b) a polynucleotide comprising a polynucleotide encoding a protein consisting of the amino acid sequence of SEQ ID NO:2;
(c) a polynucleotide comprising a polynucleotide encoding a protein consisting of the amino acid sequence of SEQ ID NO:2 with one or more amino acids thereof being deleted, substituted, inserted and/or added, and having an dihydroxy-acid dehydratase activity;
(d) a polynucleotide comprising a polynucleotide encoding a protein having an amino acid sequence having 60% or higher identity with the amino acid sequence of SEQ ID NO:2, and having an dihydroxy-acid dehydratase activity;
(e) a polynucleotide comprising a polynucleotide which hybridizes to a polynucleotide consisting of a nucleotide sequence complementary to the nucleotide sequence of SEQ ID NO:1 under stringent conditions, and which encodes a protein having an dihydroxy-acid dehydratase activity; and
(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having a dihydroxy-acid dehydratase activity.

2. The polynucleotide of claim 1 selected from the group consisting of:
(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has an dihydroxy-acid dehydratase activity;
(h) a polynucleotide encoding a protein having 90% or higher identity with the amino acid sequence of SEQ ID NO: 2, and having an dihydroxy-acid dehydratase activity; and
(i) a polynucleotide which hybridizes to SEQ ID NO: 1 or which hybridizes to a nucleotide sequence complementary to the nucleotide sequence of SEQ ID NO: 1 under stringent conditions, and which encodes a protein having an dihydroxy-acid dehydratase activity.

3. The polynucleotide of claim 1 comprising a polynucleotide consisting of SEQ ID NO: 1.

4. The polynucleotide of claim 1 comprising a polynucleotide encoding a protein consisting of SEQ ID NO: 2.

5. The polynucleotide of claim 1, wherein the polynucleotide is DNA.

6. A protein encoded by the polynucleotide of claim 1.

7. A vector comprising the polynucleotide of claim 1.

8. A yeast comprising the vector of claim 7.

9. The yeast of claim 8, wherein a capability of producing total vicinal diketones or an capability of producing total diacetyl is reduced by introducing the vector of claim 7.

10. The yeast of claim 8, wherein an capability of producing total vicinal diketones or an capability of producing total diacetyl is reduced by increasing an expression level of the protein of encoding by the polynucleotide.

11. A method for producing an alcoholic beverage comprising culturing the yeast of claim 8.

12. The method for producing an alcoholic beverage of claim 11, wherein the brewed alcoholic beverage is a malt beverage.

13. The method for producing an alcoholic beverage of claim 11, wherein the brewed alcoholic beverage is wine.

14. An alcoholic beverage produced by the method of claim 11.

15. A method for assessing a test yeast for its capability of producing total vicinal diketones or capability of producing total diacetyl, comprising using a primer or a probe designed based on a nucleotide sequence of an dihydroxy-acid dehydratase gene having the nucleotide sequence of SEQ ID NO: 1.

16. A method for assessing a test yeast for its capability of producing total vicinal diketones or capability of producing total diacetyl, comprising: culturing a test yeast; and measuring an expression level of an dihydroxy-acid dehydratase gene having the nucleotide sequence of SEQ ID NO: 1.

17. A method for selecting a yeast, comprising: culturing test yeasts; quantifying the protein according to claim 6 or measuring an expression level of an dihydroxy-acid dehydratase gene having the nucleotide sequence of SEQ ID NO: 1; and selecting a test yeast having said protein amount or said gene expression level according to a target capability of producing total vicinal diketones or capability of producing total diacetyl.

18. The method for selecting a yeast according to claim 17, comprising: culturing a reference yeast and test yeasts; measuring an expression level of an dihydroxy-acid dehydratase gene having the nucleotide sequence of SEQ ID NO: 1 in each yeast; and selecting a test yeast having the gene expressed higher than that in the reference yeast.

19. The method for selecting a yeast according to claim 17, comprising: culturing a reference yeast and test yeasts; quantifying the protein encoded by the polynucleotide in each yeast; and selecting a test yeast having said protein for a larger amount than that in the reference yeast.

20. A method for producing an alcoholic beverage comprising: conducting fermentation for producing an alcoholic beverage using the yeast according to claim 8 or a yeast selected by the method according to claim 17; and reducing the production amount of total vicinal diketones or the production amount of total diacetyl.
